(19) United States
(12) Patent Application Publication
(10) Pub. No.: US 2010/0145305 A1
(43) Pub. Date: Jun. 10, 2010

(54) LOW VOLUME ACCURATE INJECTOR

(76) Inventor: Ruth ALON, Michmoret (IL)

Correspondence Address: SUGHRUE MION, PLLC, 2100 PENNSYLVANIA AVENUE, N.W., SUITE 800, WASHINGTON, DC, 20037, US

(21) Appl. No.: 12/615,828

(22) Filed: Nov. 10, 2009

Related U.S. Application Data
(60) Provisional application No. 61/198,906, filed on Nov. 10, 2008.

(51) Int. Cl. A61M 5/31

(52) U.S. Cl. 604/506; 604/207

(57) ABSTRACT

Substance-administration apparatus is provided, including a housing and a needle couplable to the housing, having a skin-insertion portion 0.3-2.5 mm in length and an outer diameter that is less than 0.23 mm. The apparatus also comprises a dosage regulator coupled to the housing, which regulates a dosage of the substance injected in a single injection through the needle. The dosage is selectable in accordance with a setting of the dosage regulator to be one of a plurality of selectable dosages. The plurality of selectable dosages includes at least one dosage that is less than or equal to 10 ul. The apparatus further includes an injection driver, activatable by a user to drive the substance through the needle. Other embodiments are also described.

### CROSS-REFERENCES TO RELATED APPLICATIONS

The present invention claims the priority of U.S. Provisional Patent Application 61/198,906 to Alon entitled, “Low volume accurate injector,” filed Nov. 10, 2008, which is incorporated herein by reference.

### FIELD OF THE INVENTION

Embodiments of the present invention relate generally to substance administration, and specifically to apparatus and methods for administering cosmeceuticals and other drugs.

### BACKGROUND OF THE INVENTION

Mesotherapy is described by Wikipedia as a non-surgical cosmetic medicine treatment. Mesotherapy employs multiple injections of pharmaceutical and homeopathic medications, plant extracts, vitamins, and other ingredients into the subcutaneous fat.

WO 08/057,976 to Sibbitt et al. describes multiple dose syringe apparatus and methods which are described as being suitable for use to administer multiple small doses of drugs particularly for dermatology, plastic surgery, cosmetic surgery, and neurological medicine. The multiple dose syringes can be constructed from a conventional syringe and conventional plunger with adapters, and also by individual injection mounding.

WO 06/102676 to Perez et al. describes injectable implants that are described as being useful in supplementing soft tissue, particularly skin. The publication relates to dermal filler compositions of biocompatible polyethylene oxides that may be introduced, perhaps by injection, into areas of soft tissue often considered in need of augmentation. Examples include cosmetic enhancement or correction of facial defects due to scarring, aging and the like.

U.S. Pat. No. 3,794,028 to Mueller et al. describes a method of depilation in a human by injecting a dose of chemical depilatory solution into a hair follicle to permanently destroy hair growth at that location. Injection of the chemical depilatory solution may be effected by means of a hypodermic syringe for penetrating beneath the skin surface and for dispensing effective dosage amounts of the depilatory solution into the follicle.

U.S. Pat. No. 5,366,498 to Brannan et al. describes a device for correcting fine superficial facial lines, which comprises a syringe fitted with a 31-33 gauge needle and an aqueous suspension of non-crosslinked fibrillar atelopeptide collagen contained within the syringe barrel, the concentration of collagen in the suspension being in the range of 10 to 50 mg/ml, and the suspension exhibiting an extrusion plot in which there is a smooth substantially linear increase in force up to a substantially constant force in the range of 5 to 30 newtons.

U.S. Pat. No. 6,689,118 to Alchas et al. describes a method of performing an intradermal injection using a drug delivery device containing the substance to be injected. A device for practicing the method includes a needle cannula having a forward tip and a limiter portion having a skin engaging surface surrounding the needle cannula. The needle cannula is in fluid communication with the substance and the tip of the needle cannula extends beyond the skin engaging surface a distance equal to approximately 0.5 mm to 3.0 mm. The needle cannula includes a fixed angle of orientation relative to the plane of the skin engaging surface. The skin engaging surface limits penetration of the needle tip into the skin so that the substance can be expelled through the needle tip into the dermis layer. Preferably, the fixed angle of orientation of the needle cannula is generally perpendicular relative to the skin surface, and the skin engaging surface is generally flat.

US Patent Application Publication 2008-0262436 to Olson describes an injection device comprising a tubular elongated main body, a needle shield slidably arranged in said main body, a needle shield link slidably connected to said needle shield, a enclosure containing medicament arranged in said main body, a needle connected to said enclosure, a plunger operatively arranged to said enclosure for ejecting said medicament through said needle and arranged on its upper part with a number of outwardly extending stop members, spring means arranged to said plunger for operating said plunger, a dose activating means, a needle shield spring surrounding the needle shield link. The device is characterised in that said injection device further comprises a first tubular member rotationally and slidably arranged inside said needle shield link, said tubular member comprises a number or ridges and protrusions on both its outer and inner surfaces, said ridges and protrusions on the outer surface of the tubular member co-operate with guide members arranged on the inner surface of said needle shield link, said ridges and protrusions on the inner surface of the tubular member co-operate with the outwardly extending stop members of the plunger that said injection device further comprises a second tubular member arranged inside said housing, arranged and designed with a number of ridges and protrusions on its inner and outer surfaces capable of setting and delivering a certain preset dose.

U.S. Pat. No. 7,364,570 to Gerondale et al. describes a controlled volume injection/aspiration device includes a syringe having a body for containing a medicament, a needle and a piston slidably disposed within the body. A shell is provided for receiving the syringe body and a plunger rack is disposed within the shell. A manually operated control is disposed in an operative relationship with the plunger rack for moving the plunger rack in a stepwise forward direction causing the piston to eject discrete doses of medication from the syringe body through the needle. The manual operated control is also operative for moving the piston in a stepwise reverse direction causing the piston to aspirate fluid into the syringe body through the needle.

The following references may be of interest:

US 2008/0021439 to Brittingham et al.

WO 08/072,229 to Levin et al.

An insulin pen is described by Wikipedia as an insulin injection system for the treatment of diabetes. A pen has a disposable needle, a vial of insulin, and the pen housing. To use a pen, the user screws on a new needle, turns a dial on the end of the pen to the number of units of insulin needed, and inserts the needle into the skin. She then presses a button on the end of the pen to deliver the selected dose, waits until the dose is delivered, and removes the needle.

### SUMMARY OF EMBODIMENTS OF THE INVENTION

In some embodiments of the present invention, a hand-held device is provided which comprises a dosage regulator for regulating the dosage and speed of delivery of substances such as drugs, vitamins, amino acids, collagen, Botox, viscous substances, and/or other substances to a treatment site on skin of a subject. The hand-held device is couplable to or is coupled to a needle which has a skin-insertion portion that is 0.3-2.5 mm in length, and an outer diameter that is less than 0.23 mm. The hand-held device accommodates variously sized cartridges containing various substances. Expulsion of the substances from the cartridge is controlled by the dosage regulator, which is actuated by a user. The dosage regulator typically comprises an electronic dosage regulator. Alternatively, the dosage regulator comprises a mechanical dosage regulator.

The dosage is user-selectable by the user, who adjusts a setting of the dosage regulator. The dosage regulator provides a plurality of possible settings of the dosage, including at least one dosage that is less than or equal to 10 ul (microliters), and typically other dosages that are higher than this value. A user-activatable injection driver drives the substance through the needle. Additionally, the hand-held device accommodates variously sized cartridges containing various substances.

There is therefore provided, in accordance with an embodiment of the present invention, substance-administration apparatus, including:

a housing;

a needle, couplable to the housing, having a skin-insertion portion 0.3-2.5 mm in length and an outer diameter that is less than 0.23 mm;

a dosage regulator coupled to the housing, which regulates a dosage of the substance injected in a single injection through the needle, the dosage being selectable in accordance with a setting of the dosage regulator to be one of a plurality of selectable dosages, the plurality of selectable dosages including at least one dosage that is less than or equal to 10 ul; and

an injection driver, activatable by a user to drive the substance through the needle.

In an embodiment, the skin-insertion portion is 1-2.5 mm in length.

In an embodiment, the apparatus is generally cylindrical.

In an embodiment, the plurality of selectable dosages includes at least one dosage that is greater than 10 ul. The plurality of selectable dosages typically does not include any dosages that are greater than 50 ul.

In an embodiment, the at least one dosage is 5-10 ul and/or 1-5 ul.

In an embodiment, the outer diameter of the needle is less than 0.21 mm, but is typically greater than 0.15 mm.

In an embodiment, the apparatus includes a cartridge, pre-filled with the substance, couplable to the housing, for providing the substance in a plurality of injections.

In an embodiment, the needle is couplable to the housing by being couplable to the cartridge, and the needle and the cartridge are couplable to the housing by the user, prior to use of the apparatus.

In an embodiment, the substance includes a depilatory agent.

There is further provided, in accordance with an embodiment of the present invention, a substance-administration method, including:

regulating a dosage of a substance by selecting one of a plurality of selectable dosages, the plurality of selectable dosages including at least one dosage that is less than or equal to 10 ul;

inserting a needle having an outer diameter that is less than 0.23 mm into skin of a subject to a depth of 0.3-2.5 mm; and

administering the substance at the selected dosage through the needle.

In an embodiment, the method includes coupling to the needle a cartridge that is pre-filled with the substance, and providing the substance from the cartridge in a plurality of injections.

In an embodiment, the needle is inserted in skin of a face of the subject and/or skin of a neck of the subject. The substance is typically administered at least five sites, of which two of the sites are separated by less than 1 mm. The substance is typically administered at a large number of sites, and many of these sites are within 1 mm of an adjacent administration site.

The present invention will be more fully understood from the following detailed description of embodiments thereof, taken together with the drawing, in which:

### BRIEF DESCRIPTION OF THE DRAWING

FIG. 1 is a schematic illustration of a hand-held device comprising a dosage regulator and a small needle, the device being configured for administration of substances to a subject, in accordance with some embodiments of the present invention.

### DETAILED DESCRIPTION OF EMBODIMENTS OF THE INVENTION

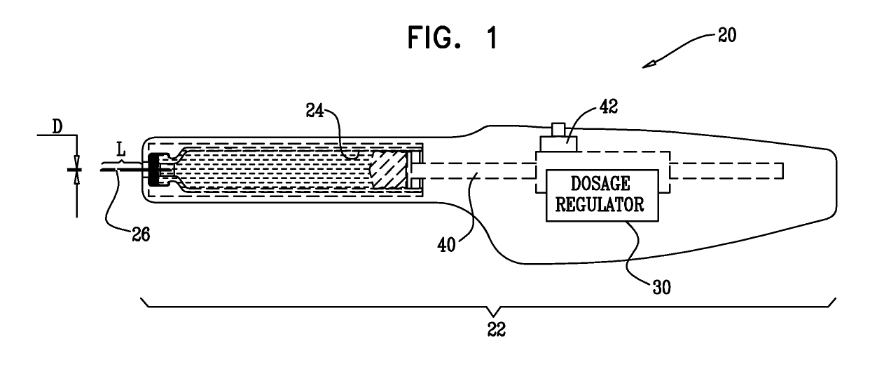

Reference is now made to FIG. 1, which is a schematic illustration of apparatus 20 for administering a substance, comprising a hand-held housing 22, a needle 26 coupled to housing 22, and, typically, a cartridge 24 containing the substance, in accordance with some embodiments of the present invention. Needle 26 has a skin-insertion portion that has a length L of 0.3-2.5 mm (e.g., 1-2.5 mm), and an outer diameter D that is less than 0.23 mm, e.g., less than 0.21 mm. For some embodiments, outer diameter D is between 0.15 mm and 0.23 mm. For example, needle 26 may comprise a 32 or 33 gauge needle, or one or more microneedles. Housing 22 is generally cylindrical. A dosage regulator 30 is coupled to the housing, and regulates a dosage of the substance injected in a single injection through needle 26. The dosage is user selectable by a user, who adjusts a setting of the dosage regulator. The dosage regulator provides a plurality of possible settings of the dosage. The dosage range includes at least one dosage that is less than or equal to 10 ul (microliters), e.g., 1-5 ul or 5-10 ul, but may also include dosages that are higher than this value, e.g., greater than 10 ul. Typically, the plurality of selectable dosages does not include any dosages that are greater than 50 ul. Dosage regulator 30 typically comprises an electronic dosage regulator and an electronic panel which displays to the user information relating to the dosage. In such an application, dosage regulator 30 comprises a user interface for the user to set the dosage and the speed of delivery of the dosage. Alternatively, dosage regulator 30 comprises a mechanical dosage regulator, typically comprising a knob, a button, or a gear system for the user to manually set the dosage and the speed of delivery of the dosage.

An injection driver 40 is activatable by the user to drive the substance through needle 26 when the user activates apparatus 20 via a user interface 42, e.g., a button. Typically, dosage regulator 30 is coupled to driver 40 and regulates (1) the speed of movement of driver 40 and (2) the distance driver 40 moves with each injection in order to control the dosage delivered to the treatment site with each injection. For some embodiments, user interface 42 is coupled to dosage regulator 30 and activates dosage regulator 30.

For some embodiments of the present invention, a pre-filled cartridge 24 is couplable to housing 22 by being fed (e.g., slid) into an opening at a distal end of housing 22 (the end of housing 22 that is closest to the skin of the user during the injection of the substance). Typically, the pre-filled cartridge 24 provides the substance to the user in a plurality of injections, e.g., for administration at least 5 sites of tissue of a subject being administered the substance. For some embodiments, two injection sites are separated by less than 1 mm.

Alternatively, cartridge 24 is coupled to housing 22 without having been pre-filled with the substance. Prior to use, cartridge 24 is loaded with the substance to be administered, e.g., by drawing proximally injection driver 40.

For some embodiments, prior to use of apparatus 20, cartridge 24 is couplable to needle 26 at a distal end of cartridge 24 (e.g., when needle 26 punctures a septum of cartridge 24), and then cartridge 24 and needle 26 are couplable to (e.g., slid into) housing 22 by the user, prior to use of apparatus 20.

As appropriate for a given application and administered substance, the dosage range may include at least one dosage that is 5-10 ul and/or at least one dosage that is 1-5 ul.

Injection driver 40 may comprise any user-activatable apparatus (e.g., a plunger, as shown) suitable for initiating and/or driving the movement of a substance through a needle into tissue of the subject.

The substance typically comprises one or more components for esthetic or medical treatment of the skin or near underlying tissue, such as adipose tissue. Apparatus 20 is particularly suitable for providing treatments to the face and neck, where known therapies (e.g., mesotherapy) have not provided satisfactory solutions in terms of dosage accuracy and minimization of pain. In particular, the needles in typical mesotherapy guns range from 30 gauge (0.305 mm outer diameter) to 26 gauge (0.457 mm), and frequently provide per-injection dosages of even as high as 100-200 ul. Mesotherapy guns are additionally generally too heavy and bulky to facilitate easy, accurate placement of the needle at a range of sites on the face or neck in close proximity to each other. The large dosages and large needles used in mesotherapy frequently result in leakage of the administered substance out of the injection site, meaning that control of the administered dosage is suboptimal.

Some embodiments of the present invention solve the problem of the inability of the prior art to adequately treat the face and neck by providing a device that is, for example, similar in general shape and user interface to an insulin pen, but with a significantly shorter needle than is used in an insulin pen. It is noted that, fundamentally, an insulin pen is designed to facilitate systemic delivery of a drug, and therefore has a long needle length, e.g., 5-12 mm. By contrast, in these embodiments of the present invention, the goal is to treat the upper layers of the skin—typically, within 0.3-2.0 mm of the surface, and therefore these embodiments of the invention utilize a significantly shorter needle, e.g., 0.3-2.5 mm in length.

As appropriate for a given application, the administered substance may comprise one or more of the following:

hyaluronic acid;

one or more vitamins (e.g., C, B10, B8, B9, I, B3 B6, B2, E, A, B12);

one or more minerals (e.g., calcium chloride, potassium chloride, magnesium sulphate, sodium acetate, sodium chloride, sodium dihydrogen phosphate);

one or more nucleic acids (e.g., deoxyadenosine, deoxycytidine, deoxyguanosine, deoxythymidine, methylcytosine);

one or more amino acids (e.g., Alainin, Arginine, Asparagine, Aspartic acid, Cystein, Glutamin, glutamic acid, glycine, histine, hydroxyproline, isoleucin, Leucine, Lysine, Methionine, Ornithine, Phenylalanine, Prolin, Serine, Taurine, Threonin, Tryptophan, Tyrosine, Valine);

one or more coenzymes (e.g., cocarboxylase, coenzyme A, flavin adenine dinucleotide phosphate, nicotinamide adenine dinucleotide, nicotinamide adenine dinucleotide phosphate, uridine triphosphate);

one or more reducing agents (e.g., ascorbic acid, glutathione);

Botox;

Collagen (cross-linked and not cross-linked);

growth hormone at homeopathic concentrations or greater; and

other hormones at homeopathic concentrations or greater.

In some embodiments, apparatus 20 described hereinabove is used to administer a depilatory agent, such as caustic soda, directly into a hair follicle. Advantageously, the small needle diameter D and length L provided by these embodiments of the invention (e.g., 32 or 33 gauge, 0.3-2.5 mm in length) allows relatively painless and accurate administration of the depilatory agent.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Substance-administration apparatus, comprising:
a housing;
a needle, couplable to the housing, having a skin-insertion portion 0.3-2.5 mm in length and an outer diameter that is less than 0.23 mm;
a dosage regulator coupled to the housing, which regulates a dosage of a substance injected in a single injection through the needle, the dosage being selectable in accordance with a setting of the dosage regulator to be one of a plurality of selectable dosages, the plurality of selectable dosages including at least one dosage that is less than or equal to 10 ul; and
an injection driver, activatable by a user to drive the substance through the needle.

2. The apparatus according to claim 1, wherein the skin-insertion portion is 1-2.5 mm in length.

3. The apparatus according to claim 1, wherein the apparatus is generally cylindrical.

4. The apparatus according to claim 1, wherein the plurality of selectable dosages includes at least one dosage that is greater than 10 ul.

5. The apparatus according to claim 1, wherein the plurality of selectable dosages does not include any dosages that are greater than 50 ul.

6. The apparatus according to claim 1, wherein the at least one dosage is 5-10 ul.

7. The apparatus according to claim 1, wherein the at least one dosage is 1-5 ul.

8. The apparatus according to claim 1, wherein the outer diameter of the needle is less than 0.21 mm.

9. The apparatus according to claim 1, wherein the outer diameter of the needle is between 0.15 mm and 0.23 mm.

10. The apparatus according to claim 1, further comprising a cartridge, pre-filled with the substance, couplable to the housing, for providing the substance in a plurality of injections.

11. The apparatus according to claim 10, wherein the needle is couplable to the housing by being couplable to the cartridge, and wherein the needle and the cartridge are couplable to the housing by the user, prior to use of the apparatus.

12. The apparatus according to claim 1, wherein the apparatus comprises the substance, and wherein the substance comprises a depilatory agent.

13. A substance-administration method, comprising:
regulating a dosage of a substance by selecting one of a plurality of selectable dosages, the plurality of selectable dosages including at least one dosage that is less than or equal to 10 ul;
inserting a needle having an outer diameter that is less than 0.23 mm into skin of a subject to a depth of 0.3-2.5 mm; and
administering the substance at the selected dosage through the needle.

14. The method according to claim 13, wherein inserting the needle comprises inserting the needle to a depth of 1-2.5 mm.

15. The method according to claim 13, wherein the plurality of selectable dosages further includes at least one dosage that is greater than 10 ul, and wherein selecting comprises selecting the at least one dosage that is greater than 10 ul.

16. The method according to claim 13, wherein the plurality of selectable dosages does not include any dosages that are greater than 50 ul, and wherein selecting comprises selecting a dosage from the plurality of selectable dosages that does not include any dosages that are greater than 50 ul.

17. The method according to claim 13, wherein the at least one dosage is 5-10 ul, and wherein selecting comprises selecting the at least one dosage that is 5-10 ul.

18. The method according to claim 13, wherein the at least one dosage is 1-5 ul, and wherein selecting comprises selecting the at least one dosage that is 1-5 ul.

19. The method according to claim 13, wherein the outer diameter of the needle is less than 0.21 mm, and wherein inserting the needle comprises inserting the needle that has an outer diameter that is less than 0.21 mm.

20. The method according to claim 13, further comprising coupling to the needle a cartridge that is pre-filled with the substance, and providing the substance from the cartridge in a plurality of injections.

21. The method according to claim 13, wherein the substance includes a depilatory agent, and wherein administering the substance comprises administering the depilatory agent.

22. The method according to claim 13, wherein inserting the needle in the skin comprises inserting the needle in at least one site selected from the group consisting of: skin of a face of the subject and skin of a neck of the subject.

23. The method according to claim 22, wherein inserting the needle in the at least one selected site comprises inserting the needle and administering the substance at least 5 sites.

24. The method according to claim 22, wherein inserting the needle in the at least one selected site comprises inserting the needle at two sites separated by less than 1 mm.
